(19) United States
(12) Patent Application Publication
(10) Pub. No.: US 2020/0181065 A1
(43) Pub. Date: Jun. 11, 2020

(54) N-ALKYLDIAMIDE COMPOUNDS AND GELS COMPRISING THE SAME

(71) Applicant: RHODIA OPERATIONS, Paris (FR)

(72) Inventors: Roland JACQUOT, Francheville (FR); Philippe MARION, Vernaison (FR); Pascal HERVE, Talence (FR); Olivier BACK, Lyon (FR); Amit SEHGAL, Potomac, MD (US)

(21) Appl. No.: 16/091,199

(22) PCT Filed: Apr. 4, 2017
(86) PCT No.: PCT/EP2017/058040

§ 371 Date: Oct. 4, 2018

Related U.S. Application Data
(60) Provisional application No. 62/318,921, filed on Apr. 6, 2016.

(51) Int. Cl. C07C 231/10; H01M 6/22

(52) U.S. Cl. CPC C07C 231/10; C07C 233/05; H01M 6/22

(57) ABSTRACT

The present invention is directed to N-alkyldiamide compounds responding to the following formula (I), wherein: R¹ or R² is selected from hydrogen or a linear, branched or cyclic, saturated or unsaturated, hydrocarbon chain having from 1 to 40 carbon atoms, with the proviso that one and only one of R¹ or R² is hydrogen, R is selected from cyclic or branched, saturated or unsaturated, hydrocarbon aliphatic chain having from 2 to 15 carbon atoms. The present invention also relates to the use of the com pounds of formula (I) as a gelling agent and to gel compositions comprising said compounds of formula (I).

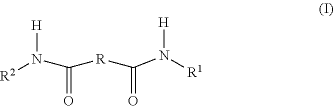

This application claims priority to U.S. provisional application No. 62/318921 filed on Apr. 6, 2016, the whole content of this application being incorporated herein by reference for all purposes.

### TECHNICAL FIELD

The present invention relates to N-alkyldiamide compounds, their synthesis and their use as structuring agents for oil and/or solvent based compositions. The present invention is also directed to oil and/or solvent based compositions thickened or gelified owing to said N-alkyldiamide compounds.

### BACKGROUND ART

Numerous fields use non aqueous oil or solvent based formulations in a thickened or gelled form as required by the application from paints, inks, agricultural actives formulations, to make up removers, lubricants and greases, industrial cleaning gels, cables filling compounds . . . In each of these applications one must adjust formulations to optimize their rheological properties.

To thicken or gelify oils or non-aqueous solvents, it is known to those skilled in the art that organogellants or low molecular weight organic gelators may be used because of their ability to self-assemble into entangled three-dimensional (3D) network structures driven by multiple, weak intermolecular forces such as π-π stacking, van der Waals, electrostatic, metal coordination, charge transfer, and H bonding interactions.

Many such gelling agents (also named gelators) have been developed and described in the literature.

EP 2 254 126 describes organogelator compounds which can be used in combination with an oil in order to form a dielectric fluid used in electric cable. The organo-gelator compounds used in this document may be selected from urea-based compounds, amide-based compounds or mixtures thereof. This document only illustrates the efficiency of urea-based compounds or of aromatic tri-amides as organogelator. EP 2 254 126 does not describe the specific N-alkyldiamide compounds such as claimed and used in the present invention.

The present invention aims at providing new compounds with remarkable gelling properties which are useful in many applications.

Unexpectedly, the inventors have discovered that the asymmetric N-alkyl diamide compounds under consideration according to the invention are gelling agents of choice.

### SUMMARY OF THE INVENTION

A first object of the invention is a compound of formula (I):

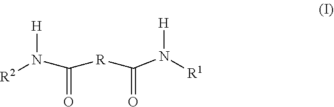

wherein:

R¹ or R² is selected from hydrogen or a linear, branched or cyclic, saturated or unsaturated, hydrocarbon chain having from 1 to 40 carbon atoms, with the proviso that either R¹ or R² is hydrogen,

R is selected from cyclic or branched, saturated or unsaturated, hydrocarbon aliphatic chain having from 2 to 15 carbon atoms.

According to an embodiment of the invention, R is selected from hydrocarbon aliphatic chains comprising a hydrocarbon main chain having from 1 to 14 carbon atoms and a side chain having from 1 to 6 carbon atoms, preferably from hydrocarbon aliphatic chains comprising a hydrocarbon main chain having from 2 to 8 carbon atoms and a side chain having from 1 to 4 carbon atoms.

According to an embodiment of the invention, R¹ or R² is selected from a linear, branched or unbranched, saturated or unsaturated, hydrocarbon group having from 2 to 40 carbon atoms, preferably from 4 to 32 carbon atoms, more preferably from 5 to 24 carbon atoms, even more preferably from 6 to 18 carbon atoms, said hydrocarbon group being preferably an aliphatic group.

Another object of the invention is a process for manufacturing the compound of formula (I) according to the invention, said process comprising the reaction between an alkylamine and a reactant selected from an imide, a diacid, a diester, a primary diamide, an ester amide, an acid ester.

According to an embodiment of the invention, the reaction between at least one alkylamine of formula (II)

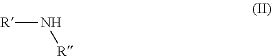

and at least one reactant selected from:
a cyclic imide of formula (III):

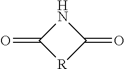

a diacid of formula (IV):

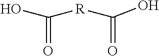

a diester of formula (V):

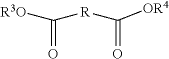

a primary diamide of formula (VI):

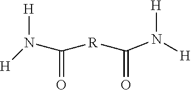

an ester amide of formula (VII):

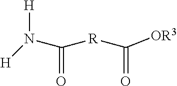

an acid ester of formula (VIII):

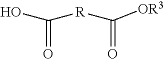

wherein:

R has the same meaning as above in formula (I),

R′ is either R¹ or R², R¹ and R² having the same meaning as above in formula (I),

R″ is either hydrogen or a hydrocarbon chain having from 1 to 40 carbon atoms,

R³ and R⁴ independently to each other, represent a hydrocarbon aliphatic chain, saturated or unsaturated, linear or branched, having from 1 to 40 carbon atoms.

Another object of the invention is a mixture comprising at least one compound of formula (I) according to the invention and at least one solvent, said compound of formula (I) being partially or fully solubilized in said solvent [hereinafter, mixture (S)].

According to an embodiment of the invention, the solvent is a polar solvent preferably selected from alcohols having from 1 to 6 carbon atoms (such as methanol, ethanol, isopropanol, butanol, isobutanol, diethyleneglycol, butylene glycol and methyl propane diol), from acetal derivatives having from 2 to 12 carbon atoms, from alkyl esters having from 2 to 12 carbon atoms (such as ethyl acetate, propyl acetate, butyl acetate, hexyl acetate, octyl acetate) and from mixtures thereof.

According to an embodiment of the invention, the compound(s) of formula (I) represent(s) from 1% to 75% by weight, preferably from 10% to 50% by weight, more preferably from 15% to 30% by weight of the total weight of the mixture (S).

Another object of the invention is the use of the compound of formula (I) according to the invention or of the mixture (S) according to the invention, as a gelling agent.

A further object of the invention is a gel composition comprising a carrier and either at least one compound of formula (I) according to the invention or the mixture (S) according to the invention, said gel composition being characterized in that the compound(s) of formula (I) is (are) self-assembled in said carrier in order to provide jellification thereof.

According to an embodiment of the invention, the carrier is selected from oils (including mineral oils, vegetable oils, animal oils and synthetic oils), linear or iso-alkanes (such as linear or iso-C₄-C₁₈ alkanes), diesters having from 8 to 40 carbon atoms, glycerol, ethyl carbonate and dimethyl carbonate. Among oils, vegetable oils (such as rapeseed oil) and methylated seed oils (such as methylated soy bean oil) are preferred.

According to an embodiment of the invention, the compound(s) of formula (I) represent(s) from 0.01% to 10% by weight, preferably from 0.05% to 5% by weight, more preferably from 0.1% to 2.5% by weight, even more preferably from 0.1% to 1% by weight, of the total weight of the gel composition.

According to an embodiment of the invention, the carrier represents from 50% to 99.99% by weight, preferably from 60% to 99.95% by weight, more preferably from 70% to 99.9% by weight, even more preferably from 80% to 99.9% by weight, of the total weight of the gel composition.

According to an embodiment of the invention, the gel composition is a battery electrolyte.

According to an embodiment of the invention, the gel composition is a cosmetic product, such as a personal care product.

The N-alkyldiamide compounds of the present invention have the following advantages:
they can be used at very low concentrations,
they allow the formation of strong gel compositions,
they are multi-purposes in the sense that they allow obtaining a gelled composition based on very different kinds of carriers or solvents from non-polar to polar solvents: the use of different chain length of the gelator allows to adapt said gelator to different solvents or oils. Furthermore, the mixing of gelators with different chain lengths offers another kind of flexibility;
The use of the mixture (S) of N-alkyl diamide gelators, wherein the gelators are partially or fully in a solubilized form, allow for the gelation at room temperature with simple mixing.

In particular, the gelled compositions obtained owing to the N-alkyldiamide compounds of the invention are very stable, they keep their gel form for a long time, up to relatively high temperatures. The gelled compositions according to the invention show no syneresis phenomenon for most solvents.

The gelled compositions according to the invention show large elastic and storage moduli even at very low gelator concentrations.

### DETAILED DESCRIPTION OF THE INVENTION

### N-alkyldiamide Compound

The present invention is directed to a compound responding to the following formula (I):

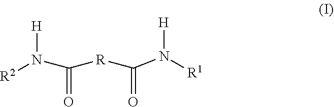

wherein:

R¹ or R² is selected from hydrogen or a linear, branched or cyclic, saturated or unsaturated, hydrocarbon chain having from 1 to 40 carbon atoms, with the proviso that one and only one of R¹ or R² is hydrogen,

R is selected from cyclic or branched, saturated or unsaturated, hydrocarbon aliphatic chain having from 2 to 15 carbon atoms.

Thus, in the compound of formula (I) above, R¹ or R² is hydrogen being understood that if R¹ is hydrogen, then R² cannot be hydrogen and if R² is hydrogen, R¹ cannot be hydrogen.

Within the meaning of the present invention, by “hydrocarbon chain”, it is to be understood a hydrocarbon chain comprising carbon atoms and hydrogen atoms, wherein said hydrocarbon chain may optionally be substituted by one or more heteroatoms, such as oxygen atoms.

Within the meaning of the present invention, by “aliphatic chain”, it is to be understood a non-aromatic chain.

According to an embodiment of the invention, R¹ or R² of the compound of formula (I) is a hydrocarbon chain constituted only by carbon atoms and hydrogen atoms.

According to an embodiment of the invention, R¹ or R² of the compound of formula (I) is aromatic. In particular, R¹ or R² may be selected from phenyl or furyl groups.

According to another embodiment of the invention, R¹ or R² of the compound of formula (I) is a linear, branched or cyclic aliphatic (non-aromatic) hydrocarbon chain.

According to an embodiment of the invention, R¹ or R² is selected from a linear or branched, saturated or unsaturated, hydrocarbon chain having from 2 to 40 carbon atoms, preferably from 4 to 32 carbon atoms, more preferably from 5 to 24 carbon atoms, even more preferably from 6 to 18 carbon atoms.

According to an embodiment of the invention, R¹ or R² is selected from a linear, unbranched, saturated or unsaturated, hydrocarbon aliphatic chain having from 2 to 40 carbon atoms, preferably from 4 to 32 carbon atoms, more preferably from 5 to 24 carbon atoms, even more preferably from 6 to 18 carbon atoms.

According to an embodiment of the invention, R¹ or R² is selected from a linear unbranched saturated hydrocarbon aliphatic chain having from 2 to 40 carbon atoms, preferably from 4 to 32 carbon atoms, more preferably from 5 to 24 carbon atoms, even more preferably from 6 to 18 carbon atoms.

According to an embodiment of the invention R¹ or R² is selected from pentyl, hexyl, octyl, decyl, dodecyl, tetradecyl, palmityl, stearyl, 12-hydroxy stearyl, oleyl, 12-hydroxyloleyl, linoleyl, linolenyl, arachidyl or behenyl groups.

According to an embodiment of the invention, R is not cyclic. Preferably, R is selected from hydrocarbon aliphatic chains comprising a hydrocarbon main chain having from 1 to 14 carbon atoms and a side chain having from 1 to 6 carbon atoms. Preferably, R is selected from hydrocarbon aliphatic chains comprising a hydrocarbon main chain having from 2 to 8 carbon atoms and a side chain having from 1 to 4 carbon atoms.

According to an embodiment of the invention, R is selected from —CH(CH₃)—CH₂—; —CH₂—CH(CH₃)—; —CH(CH₃)—CH₂—CH₂—; —CH(CH₂—CH₃)—CH₂— and —CH₂—CH₂—CH(CH₃)—.

Preferably, the compounds of formula (I) according to the invention do not present urea functions of type —NH—CO—NH—.

The present invention is also directed to a process for manufacturing the compound of formula (I) according to the present invention, said process comprising the reaction between at least one alkylamine and at least one reactant selected from an imide, a diacid, a diester, a primary diamide, an ester amide, an acid ester or other combinations.

Ammonia may also be added during the process if it is not already present in the molecule as amide or imide functions. Ammonia may allow favoring the manufacture of the mono-alkyl form (versus the dialkyl form) of the diamide and may also allow favoring the elimination of by-products formed during the reaction (such as alcohol coming from reaction with ester as a co-product).

General processes for manufacturing diamides are described in document WO 2010/031867.

According to an embodiment of the invention, the reaction is an amidification. According to an embodiment, the process of the invention comprises the reaction between at least one alkylamine of formula (II)

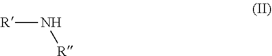

and at least one reactant selected from:
a cyclic imide of formula (III):

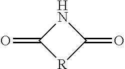

a diacid of formula (IV):

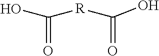

a diester of formula (V):

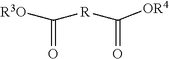

a primary diamide of formula (VI):

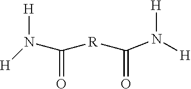

an ester amide of formula (VII):

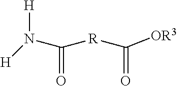

an acid ester of formula (VIII):

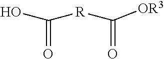

wherein:

R has the same meaning as above in formula (I),

R′ is either R¹ or R², R¹ and R² having the same meaning as above in formula (I),

R″ is either hydrogen or a hydrocarbon chain having from 1 to 40 carbon atoms,

R³ and R⁴ independently to each other, represent a hydrocarbon aliphatic chain, saturated or unsaturated, linear or branched, having from 1 to 40 carbon atoms.

In particular, the process of the invention comprises the direct reaction between a cyclic imide of formula (III) and an alkylamine of formula (II), wherein:

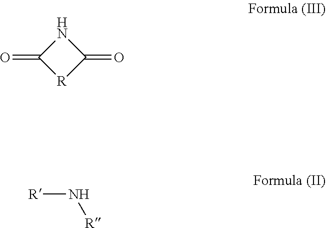

wherein:

R has the same meaning as above in formula (I),

R′ is either R¹ or R², R¹ and R² having the same meaning as above in formula (I),

R″ is either hydrogen or a hydrocarbon chain having from 1 to 40 carbon atoms.

The compound of formula (I) can thus be obtained by an imide ring-opening reaction of the cyclic imide with addition of the alkylamine.

The imide may be selected from 2-methyl-glutarimide, 3-methyl-glutarimide, 2-ethyl-glutarimide, 3-ethyl-glutarimide, 2-methyl-succinimide, 2-ethyl-succinimide.

For example, in order to obtain a compound of formula (I) wherein R is —CH₂—CH₂—CH(CH₃)— or —CH(CH₃)—CH₂—CH₂, then the imide can be a cyclic imide of formula (III-1):

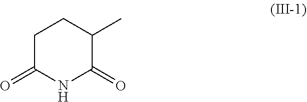

The alkylamine may be selected from primary alkylamine or secondary alkylamine. Preferably, the alkylamine has the following formula (II-1): R′NH₂ wherein R′ is R¹ or R² as defined above for the compound of formula (I).

The reaction for preparing the compounds of formula (I) according to the invention may be performed for example, at a temperature ranging from 20° C. to 200° C., preferably from 50° C. to 180° C., more preferably from 100° C. to 160° C. In the case of lighter alkylamines, such as alkylamines having less than 6 carbon atoms, the reaction may be performed with a progressive heating in order to avoid loss of amines through evaporation or may be performed under pressure to keep the amine in the liquid phase. For example, at the beginning of the reaction, the temperature of the mixture (ingredients) is approximately equal to the boiling point of the alkylamine.

The reaction for preparing the compounds of formula (I) according to the invention is preferably performed under atmospheric pressure.

The product obtained at the end of the reaction may then be recovered using purification methods well known for the skilled person, such as recrystallization methods.

The present invention is also directed to the use of at least one compound of formula (I) according to the invention as a gelling agent (gelator).

Indeed, the inventors surprisingly found that the compounds of formula (I) has remarkable gelling properties, in very different carriers. The compounds of formula (I) allows gelling different kinds of compositions, including non-polar solvents (such as mineral oils, vegetable oils, animal oils and synthetic oils) and polar solvents (such as glycerol and carbonates).

### Mixture (S) Containing N-alkyldiamide Compound(s)

The present invention is also directed to a mixture (S) comprising at least one compound of formula (I) according to the invention and at least one solvent, said compound of formula (I) being partially or fully solubilized in said solvent.

In the mixture (S) of the invention, the at least one compound of formula (I) is preferably fully solubilized in the at least one solvent. The mixture (S) is preferably a solution comprising at least one solvent and, fully dissolved therein, at least one compound of formula (I), that is to say the mixture (S) is free of any compound that would be insolubilized in the solvent.

Preferably, the mixture (S) is one which, when at room temperature and at atmospheric pressure (20° C., 1 atm), comprises the compound of formula (I) in partially or fully solubilized form in the solvent. More preferably, the mixture (S) is one which, when at room temperature and at atmospheric pressure, comprises the compound of formula (I) in fully solubilized form.

Advantageously, the mixture (S) is one which, when at 60° C. and at atmospheric pressure, comprises the compound of formula (I) in fully solubilized form in the solvent. Preferably, the mixture (S) is one which, when at 40° C. and at atmospheric pressure, comprises the compound of formula (I) in fully solubilized form.

Advantageously, the mixture (S) is a solution when it is put at 60° C. and at atmospheric pressure. Preferably, the mixture (S) is a solution when it is put at 40° C. and at atmospheric pressure. More preferably, the mixture (S) is a solution when it is put at room temperature (20° C.) and at atmospheric pressure.

With the meaning of present invention, the solubilized form is different from the self-assembled form.

According to an embodiment, the solvent used in the mixture (S) according to the invention is a good solvent and can be a polar solvent or a mixture of a polar solvent and an apolar solvent. Within the meaning of the present invention, by “polar solvents”, it is to be understood solvents having a polar and hydrogen bonding components δₚ+δₕ (Hansen solubility parameter) strictly greater than 0. Hansen solubility parameters are well known for the skilled person: δₚ corresponds to the energy from dipolar intermolecular forces between molecules and δₕ corresponds to the energy from hydrogen bonds between molecules.

According to an embodiment of the invention, the solvent of the mixture (S) is selected from alcohols having from 1 to 10 carbon atoms, preferably selected from methanol, ethanol, isopropanol, butanol, isobutanol, diethylene glycol, butylene glycol, methyl propane diol.

According to an embodiment of the invention, the solvent of the mixture (S) is selected from acetal derivatives having from 2 to 12 carbon atoms, and is preferably selected from the Solvay available Augeo® family of solvents such as Augeo® SL191 which is a racemic mixture (+/−)-2,2-dimethyl-4-hydroxymethyl-1,3-dioxolane also known as isopropylidene glycerol.

According to an embodiment of the invention, the solvent of the mixture (S) is selected from alkyl esters having from 2 to 12 carbon atoms, preferably selected from ethyl acetate, propyl acetate, butyl acetate, hexyl acetate, octyl acetate.

According to an embodiment of the invention, the solvent of the mixture (S) is itself a mixture of (i) alcohols having from 1 to 10 carbon atoms (preferably selected from methanol, ethanol, isopropanol, butanol, isobutanol, diethylene glycol, butylene glycol, methyl propane diol), (ii) alkyl esters having from 2 to 12 carbon atoms (preferably selected from ethyl acetate, propyl acetate, butyl acetate, hexyl acetate, octyl acetate) and (iii) acetal derivatives having from 2 to 12 carbon atoms (preferably selected from the Solvay available Augeo family of solvents such as Augeo® SL191 which is a racemic mixture (+/−)-2,2-dimethyl-4-hydroxymethyl-1,3-dioxolane also known as isopropylidene glycerol).

These solvents are capable of dissolving the compounds of the invention, the latter being generally in a powder form as a solid.

The mixture (S) according to the invention may be from a liquid state to a flowable thick paste at temperatures ranging from 20° C. to 50° C.

According to an embodiment of the invention, the compounds of formula (I) represents from 1% to 75% by weight, preferably from 10% to 50% by weight, more preferably from 15% to 30% by weight, of the total weight of the mixture (S).

According to a particular embodiment, the mixture (S) of the invention comprises at least two different compounds of formula (I).

According to a particular embodiment, the mixture (S) of the invention comprises more than two different compounds of formula (I).

By “different compounds of formula (I)”, it is to be understood two compounds of formula (I) wherein the groups R, R¹ or R² are different. In particular, when the mixture (S) of the invention comprises compounds differing notably by the chain length of R¹ or R², the use of the mixture (S) is more flexible and can be used in very different carriers in order to allow the gelification of said carriers.

The mixture (S) according to the invention may be obtained by simple mixing of the ingredients (compound of formula (I) and solvent), preferably at a temperature comprised between 10° C. and 60° C., ideally about 25° C. (room temperature).

The mixture (S) allows obtaining a final gelled composition more easily, in particular, the final gel composition, when starting from the mixture (S) of the invention, may be obtained by mixing the ingredients at ambient temperature.

The most important advantage of the mixture (S) of the invention is that it allows the gelification of a composition at room temperature (about 25° C.), which can be very useful or even necessary if the solvents or oils to be gelified or thickened are too volatile at the high temperature required for the mixing or if there are additives in the composition that are sensitive to high temperatures.

The mixture (S) according to the invention may be used in a composition in order to gelify said composition.

### Gel Composition Comprising the N-alkyldiamide Compound(s)

The present invention is also directed to a gel composition comprising at least one compound of formula (I) according to the present invention and at least one carrier, said compound(s) of formula (I) being in a self-assembled form in said carrier in order to provide gelification thereof.

Within the meaning of the present invention, by “self-assembled form” it is to be understood that the gelator self assembles into entangled three-dimensional (3D) network structures driven by multiple, weak intermolecular forces such as π-π stacking, van der Waals, electrostatic, metal coordination, charge transfer, and H bonding interactions.

According to an embodiment of the invention, the elastic modulus of the gel composition ranges from 0.5 Pa to 500000 Pa. The elastic modulus may be measured with a ARG2® Rheometer from TA instruments, as described in the experimental part.

According to a preferred embodiment of the invention, the compound of formula (I) represents from 0.01% to 10% by weight, preferably from 0.05% to 5% by weight, more preferably from 0.1% to 2.5% by weight, even more preferably from 0.1% to 1% by weight, of the total weight of the gel composition.

According to a preferred embodiment, the carrier represents from 50% to 99.99% by weight, preferably from 60% to 99.95% by weight, more preferably from 70% to 99.9% by weight, even more preferably from 80% to 99.9% by weight, of the total weight of the gel composition.

According to an embodiment, the carrier is selected from non-polar to polar solvents, preferably selected from mineral oils, vegetable oils, animal oils or synthetic oils. For example, the carrier may be selected from vegetable oils, such as rapeseed oil sometime known as Canola oil, methylated seed oils, such as methylated soy bean oil, linear or iso-alkanes, such as linear or iso-C₄-C₁₈ alkanes, diesters having from 8 to 40 carbon atoms, glycerol, ethyl carbonate and dimethyl carbonate.

The carrier may be selected according to the intended final use of the gelled composition. The gelled composition may be used in cosmetic formulations or in the agricultural coatings or cleaning.

According to an embodiment of the invention, the carrier is chosen among those acceptable in cosmetic formulations.

According to a particular embodiment, the gel composition of the invention comprises at least two or more different compounds of formula (I).

According to an embodiment, the gel composition of the invention further comprises at least one additional additive, preferably selected from surfactants.

These additives are selected according to the intended final use of the gelled composition.

According to this embodiment, the additional additive(s) may represent(s) from 0.1% to 45% by weight, preferably from 1% to 40% by weight, more preferably from 5% to 30% by weight, of the total weight of the gel composition.

According to an embodiment of the invention, the gel composition comprises, in particular consists in:
from 0.01% to 10% by weight, preferably from 0.05% to 5% by weight, more preferably from 0.1% to 2.5% by weight, even more preferably from 0.1% to 1% by weight, of compound(s) of formula (I) according to the invention,
from 50% to 99.99% by weight, preferably from 60% to 99.95% by weight, more preferably from 70% to 99.9% by weight, even more preferably from 80% to 99.9% by weight, of carrier(s),
up to 45% by weight, preferably from 0.1% to 45% by weight, more preferably from 1% to 40% by weight, even more preferably from 5% to 30% by weight, of additional additive(s).

The gel composition may be prepared either directly from the compound of formula (I) alone or from the mixture (S) according to the invention comprising at least one compound of formula (I).

The gel composition may be obtained by adding the compound of formula (I) (gelator) into the carrier for example at a temperature ranging from 50° C. to 150° C., preferably from 70° C. to 130° C., more preferably from 90° C. to 110° C. Preferably, the carrier is agitated. The agitation can be performed using means well known for the skilled person, such as a tip sonicator or a propeller agitation. The agitation may lapse from 1 to 10 minutes.

Then, the gel composition can be left to cool back or can be cooled for example to room temperature (about 25° C.).

The gel composition may also be obtained by adding the mixture (S) according to the invention into the carrier for example at ambient temperature or at a temperature ranging from 20° C. to 60° C., preferably from 23° C. to 30° C. Preferably, the carrier is agitated. The agitation can be performed using means well known for the skilled person, such as a tip sonicator or a propeller agitation. The agitation may lapse from 1 to 10 minutes.

The gel composition according to the invention may be used in different fields of application, such as in batteries or in personal care products.

Indeed, the gel composition according to the invention may be used as a gel electrolyte in batteries.

The gel composition according to the invention may also be used as a personal care product, in particular for cosmetic applications. Indeed, it has been found that the gelators according to the invention do not lose their gelling properties even in the presence of other functional additives such as surfactants generally found in personal care products.

Should the disclosure of any patents, patent applications, and publications which are incorporated herein by reference conflict with the description of the present application to the extent that it may render a term unclear, the present description shall take precedence.

### EXAMPLES

### Example 1: Preparation of N-alkyldiamide Compounds

### Example 1a: Preparation of a N-hexyldiamide Compound (Also Named Hereafter C6 Gelator)

In a round bottom flask equipped with a magnetic stirrer and a condenser, are added 50.0 g of a mixture composed of 2-methylglutarimide (MGI) (86.4 wt %, 0.34 mol) and 2-ethylsuccinimide (10.7 wt %, 0.04 mol), 40.1 g of hexylamine (0.40 mol) and 0.9 g of tBuONa.

The mixture is then allowed to stir at 130° C. during 2 hours. Over the course of the reaction the mixture becomes monophasic (yellow solution). The reaction progress is followed thanks to GC analysis and at the end of the reaction, the product is recovered thanks to recrystallization in 150 mL of methyl ethyl ketone (MEK) followed by washing two times with 100 mL of ethyl acetate.

After drying under vacuum, 71.5 g (92% yield based on MGI) of the product is obtained as a white powder (melting point (mp): 111.8-115.4° C.).

NMR analysis shows that the product is obtained as a mixture of 2 isomers:
N¹-hexyl-4-methylpentanediamide (57 wt %),
N¹-hexyl-2-methylpentanediamide (43 wt %).

### Example 1b: Preparation of a N-lauryldiamide Compound (Also Named Hereafter C12 Gelator)

In a round bottom flask equipped with a magnetic stirrer and a condenser, are added 17.0 g of a mixture composed of 2-methylglutarimide (86.4 wt %, 0.12 mol) and 2-ethylsuccinimide (10.7 wt %, 0.01 mol), 25.0 g of dodecylamine (0.13 mol) and 0.4 g of tBuONa.

The mixture is then allowed to stir at 150° C. during 2 hours. Over the course of the reaction the mixture turns yellow and reaction progress can be followed thanks to GC analysis. After reaction completion, the desired product is recovered thanks to recrystallization in 200 mL of methyl ethyl ketone (MEK) followed by washing two times with 100 mL of ethyl acetate.

After drying under vacuum, 29.1 g (78% yield based on MGI) of the product is obtained as a white powder (mp: 112.9-117.8° C.).

NMR analysis shows that the product is obtained as a mixture of 2 isomers:
N1-lauryl-4-methylpentanediamide (58 wt %),
N1-lauryl-2-methylpentanediamide (42 wt %).

### Example 1c: Preparation of a N-oleyldiamide Compound (Also Named Hereafter C18:1 Gelator)

In a 500 ml round bottom flask equipped with a magnetic stirrer and a condenser are added 50 g of a mixture composed of 2-methylglutarimide (86.4 wt %, 0.34 mol) and 2-ethylsuccinimide (10.7 wt %, 0.04 mol), 105 g of oleylamine (0.39 mol), 0.5 g of tBuONa. The mixture is then heated till 150° C. for 2 hours. After cooling down, we added 200 mL of THF; heat till reflux then cooled down again. Crystallisation, filtration and washing the solid with ethylacetate, drying allow to obtain 57 g of white solid. The purity of the product is >98%. The yield is only 43% but it could easily be optimized as we have recovered later 60 g more of product after second crystallization of the mother liquor.

### Example 2: Preparation of Gel Compositions

All compositions were prepared in 20 ml scintillation glass vials. Appropriate weights of the gelator powder and the oils or solvents were weighted on a microbalance. Total weights of gelators plus liquid oil or solvent were from 8 to 18 g.

For examples no 2A through 2F: Good dispersions and solubilizations of the gelators at high temperatures (90° C. to 110° C.) were obtained using a tip sonicator (Branson Sonifier® 450) 6.3 mm diameter tip at a duty cycle set at 50%, power at 70%, with sonication durations set at 4 minutes. All samples were fluid, transparent and clear at end of the sonicating preparation.

Unless specifically mentioned otherwise all samples were left to cool back to room temperature on a bench. The qualitative visual verification of the gelation was done by simply upturning the vials. The gelation was considered successful if no flow was observed upon inverting the vial at room temperature.

In the specific case of example no 2A the samples were immediately introduced upon sonication while still liquid into the cup of a ARG2® Rheometer from TA Instruments already set at 80° C. with the conical cylinder geometry quickly lowered to the appropriate gap immediately followed by a oscillatory collection of elastic modulus G′ versus time as the temperature was quenched from 80° C. to 25° C. then remaining at 25° C. using the Peltier temperature controlled stage.

### Example 2A: Gel Composition Comprising a Vegetable Oil (Canola Oil) and Either the Gelator of Example 1a (C6 gelator) or the Gelator of Example 1b (C12 Gelator)

The Wesson brand Canola oil was purchased from a retail supermarket and used for these tests. The C6 and C12 Gelator concentrations tested ranged from 0.1 to 2 wt %. Both C6 and C12 gelators display the ability to prepare gels in canola oil. In particular, strong gels in Canola may be obtained with a quantity of gelator above 0.2 wt % for the C12 gelator and above 0.3 wt % for the C6 gelator. The obtained gels resist an upside-down flip and stick to the bottom of the vial without flowing. The gels are all perfectly homogenous. The elastic modulus G′ was measured on a ARG2® Rheometer from TA Instruments using a conical cylinder geometry through the cooling step from 80° C. to 25° C. The obtained gels had G′ values ranging from 0.5 Pa to 500000 Pa for the C6 gelator in canola oil and from 20 to 20000 Pa for the C12 Gelator.

The Canola organogels obtained with low concentrations (around 0.3% wt) of C6 gelator are transparent and clear.

### Example 2B: Gel Composition Comprising a Methylated Seed Oil (Methylated Soy Bean Oil) and Either the Gelator of Example 1a (C6 Gelator) or the Gelator of Example 1b (C12 Gelator)

Methyl esters of vegetable oils also referred to as methylated seed oils are commonly used as raw material in Agricultural formulations. Gel occurs in methylated soybean oil thanks to the gelators of the invention. Strong gelations were observed in a good representative of such oils, for concentrations of 0.5% of the C6 gelator, and 1% of the C12 gelator. The obtained gels resist to an upside-down flip, and are soft, slightly cloudy.

### Example 2C: Gel Composition Comprising a Linear Alkane Solvent (Hexadecane) and Either the Gelator of Example 1a (C6 Gelator) or the Gelator of Example 1b (C12 Gelator)

Sigma-Aldrich 98% purity alkanes Hexadecane was used for gelation tests. Hexadecane gels were obtained with a concentration of C12 gelator of for example 1 wt % and with a concentration of C6 gelator of for example 3 wt %. Gels are slightly stronger.

### Example 2D: Gel Composition Comprising a Iso-Alkane Solvent (Iso-Hexadecane) and Either the Gelator of Example 1a (C6 Gelator) or the Gelator of Example 1b (C12 Gelator)

Isohexadecane (Purolan® IHD) was provided by Lanxess.

Both gelators induce gelation in Isohexadecane at a tested concentration of about 1 wt %. Visual observations indicate the gel obtained with the C6 gelator appears to be weaker than the one obtained with the C12 gelator. The gel using the C6 gelator is less cloudy than the one obtained with the C12 gelator.

### Example 2E: Gel Composition Comprising a Diester Solvent (RhodiaSolv RPDE® and RhodiaSolv IRIS®) and Either the Gelator of Example 1a (C6 Gelator) or the Gelator of Example 1b (C12 Gelator)

The obtained solutions tend to gel slowly. In RhodiaSolv RPDE® solvent (available from Solvay), a wide range of concentrations of the C12 gelator from 0.1% wt to 5% wt—was screened during the trials with repeatable gelation.

C12 gelator achieves gelation in RhodiaSolv IRIS® solvent (available from Solvay) at 1% w, and at 0.8 wt % in RhodiaSolv RPDE® solvent in a 24 hours-time. It has to be noted that the C12 gelator appeared more efficient with RhodiaSolv RPDE® solvent than with RhodiaSolv IRIS® solvent.

C6 gelator achieves gelation in RhodiaSolv RPDE® solvent (Concentration preferably higher than or equal to 1% wt) and RhodiaSolv IRIS® solvent (Concentration preferably higher than or equal to 1% wt) slowly over 1 day rather than hours.

### Example 2F: Gel Composition Comprising a Glycerol Solvent and the Gelator of Example 1b (C12-Gelator)

The C12 Gelator achieves gelation of the glycerol, for example at concentrations from 0.5 to 2 wt %. The obtained gel compositions could be compared to a petrolatum jelly for its appearance as grey viscous gels. The paste does not flow, even at long times.

### Example 3: Use of C6 Gelator (Gelator of example 1a) in an Ethanol-Based Solution (S)

A solution (S) of the C6 Gelator at 25 wt % in Ethanol was prepared. A few droplets of this solution (S) (typically 0.200 g of solution (S) in 7.800 g of the solvent or oil to be gelled) was introduced in the vials which were then capped and vortexed for 30 seconds. The sample was then left to rest on the bench. Successful gelation was recorded using this C6 Gelator solution (S) in the following carriers: Canola Oil, Soy Bean Oil, Dodecane, Hexadecane, Isododecane, Isohexadecane, Methylated Soy Bean Oil, RhodiaSolv RPDE®, Diesel and Light crude oil. An effective concentration in C6 gelator of about 0.6 wt % was tested. The use of the solution (S) eliminated the high temperature step and allowed gelation through simple mixing at room temperature (about 25° C.).

### Example 4: Use of a Combined C6 Gelator (Gelator of Example 1a) and C12 Gelator (Gelator of Example 1b) in an Ethanol-Based Solution (S)

A solution (S) of the C6 Gelator at 26.4 wt % and C12 gelator at 8.1 wt % in Ethanol was prepared. A few droplets of this solution (S) (typically 0.100 g of solution (S) in 3.900 g of the solvent or oil to be gelled) was introduced in the vials which were then capped and Vortexed for 30 seconds. The sample was then left to rest on the bench. Successful gelation was recorded using this mixed C6+C12 gelators solution (S) in the following carriers: Canola Oil, Hexadecane, Isohexadecane. An effective concentration in mixed gelator of about 0.8 wt % was tested. The use of the solution (S) eliminated the high temperature step and allowed gelation through simple mixing at room temperature (about 25° C.).

### Example 5: Use of C6 Gelator (Gelator of Example 1a) in the Form of a Solution (S) in Augeo® SL191

A solution (S) of the C6 Gelator at 28.8 wt % in Augeo® SL 191 (available from Solvay) racemic mixture (+/−)-2,2-dimethyl-4-hydroxymethyl-1,3-dioxolane was prepared. A few droplets of this solution (S) (typically 0.200 g of solution in 7.800 g of the solvent or oil to be gelled) was introduced in the vials which were then capped and Vortexed for 30 seconds. The sample was then left to rest on the bench. Successful gelation was recorded using this C6 Gelator solution (S) in the following carriers: Canola Oil, Hexadecane, Isohexadecane, Methylated Soy Bean Oil, RhodiaSolv RPDE®. An effective concentration in C6 gelator of about 0.7 wt % was tested. The use of the solution (S) eliminated the high temperature step and allowed gelation through simple mixing at room temperature (about 25° C.).

### Example 6: Use of C18:1 Gelator (Gelator of Example 1c) in the Form of a Solution (S) in a Mixture of octylacetate and methylpropanediol

A solution (S) of 20 wt % of the C18:1 Gelator, 60 wt % octyl acetate and 20 wt % methyl propanediol was prepared. A few droplets of this solution (S) (typically 0.400 g of solution (S) in 7.600 g of the solvent or oil to be gelled) was introduced in the vials which were then capped and Vortexed for 30 seconds. The sample was then left to rest on the bench. Successful gelation was recorded using this C18:1 Gelator solution (S) the following carriers: Canola Oil, Grape Seed Oil, Light Mineral Oil. An effective concentration in C18:1 Gelator of about 1 wt % was tested. The use of the solution (S) eliminated the high temperature step and allowed gelation through simple mixing at room temperature (about 25° C.).

### Example 7: Effect of the Addition of a Surfactant

### Example 7a: Canola Oil Gel Compositions

Canola oil gel compositions obtained with 0.5 wt % of the C6 Gelator remained in the form of a gel upon the addition of one of the following freeze-dried surfactants:

a) 1.5 wt % of Rhodapex® (sodium alkyl sulfate available from Solvay)
b) 4 wt % of Mirataine® BET-C30 (cocamidopropyl betaine available from Solvay)
c) 5 wt % Alkamuls® SMO (sorbitan monooleate available from Solvay)

### Example 7b: Methylated Soy Bean Oil Gel Compositions

Methylated soybean oil gel compositions prepared with 1 wt % of the C6 gelator remained in the form of a gel with the addition of up to 10 wt % of the original and the freeze-dried surfactant blend AgRho® EM30 (available from Solvay).

Methylated Soy Bean Oil Gels prepared with 1 wt % of the C12 gelator seemed to hold up to 20% of original AgRho Blend.

### Example 8: Personal Care Cleansing Formulations

A gel composition was prepared containing 2 wt % of C6 Gelator, 92.85 wt % of Ethylhexyl Palmitate, 5.15 wt % of Baby Oil and displayed a G′ elastic modulus of 10000 Pa.

A gel was maintained after adding 5 wt % of Alkamuls® SMO (sorbitan monooleate) to the composition prepared above leading to a composition containing 1.9 wt % of C6 Gelator, 88.43 wt % of ethylhexyl palmitate, 4.9 wt % of Baby Oil, 4.76 wt % Alkamuls SMO. It resulted in a softer gel having a G′ elastic modulus of 250 Pa.

### Example 9: Gel of a Battery Electrolyte

Gel compositions of a typical battery electrolyte made of an equal volume of Ethyl Carbonate and Dimethyl Carbonate were prepared using 2 wt % of the C12 Gelator.

Examples 1 to 9 show that the claimed compounds have very satisfying gelling properties in very different compositions. In particular, the gelling properties are not altered by the presence of a functional additive, such as a surfactant. Furthermore, it has been found that the compounds according to the invention have gelling properties in compositions used in gel battery electrolyte compositions.

### Example 10: Comparative Diamide Compounds

### Example 10a: N1,N5-dihexyl-2-methylpentanediamide

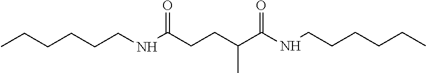 [SMILES: CCCCCCNC(=O)CCC(C)C(=O)NCCCCCC]

N1,N5-dihexyl-2-methylpentanediamide can be prepared by full amidation of MGDC (methyl-2glutaroyldichloride) by n-hexylamine.

In a round bottom flask equipped with a magnetic stirrer and a condenser, were added 0.12 mol of 2-methylglutaroyldichloride (22 g) and 30.3 g of n hexylamine (0.3 mol) and 0.36 mol—36.4 g—of triethylamine.

The mixture was then allowed to stir at 30° C. during 6 hours. After reaction completion, the desired product was recovered thanks to filtration. The crude white solid was recrystallised in ethyl acetate (100 mL) to get after drying 30 g of a white solid (purity>98%) corresponding to a yield of 80%

Compositions of 1 wt % and 2 wt % of N1,N5-dihexyl-2-methylpentanediamide in Rapeseed oil were prepared according to the same protocol as described above (example 2). Both samples became transparent and clear sonication duration was 4 minutes. No Gel formation was observed for either after 24 hours and up to a week after sample preparation. Both samples remained liquid.

### Example 10b: N1-dodecyl-adipamide

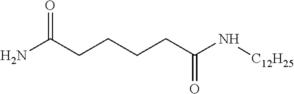

N1-dodecyl-adipamide can be prepared by coupling commercial cyanovaleramide (11.6 g) with laurylamine (14.8 g) in presence of conventional coupling agent EDC,HCl (N-(3-Dimethylaminopropyl)-N′-ethylcarbodiimide hydrochloride—1.2 equ.), hydroxybenzotriazole (HOBt, 1.3 equ.) and diisopropylethylamine (DIPEA, 5 equ.) in a solvent mixture DMF/THF (200 ml/400 ml). After 18 h of stirring at room temperature, the suspended mixture was filtrated. The solid was washed with water then dried to afford a crude white solid. Further washings after agitation in THF, then n-hexane then drying allow to obtain a 15.2 g of a pure product (Yield 61%).

Compositions of 1 wt % and 2 wt % of N1-dodecylAdipamide in Rapeseed oil were prepared according to the same protocol as described above (example 2). Neither samples became transparent and clear after 4 minutes of sonication even extended to 10 minutes. No Gel formation was observed for either after 24 hours and up to a week after sample preparation. Samples were both phase separated after 24 hours for the 2 % wt sample and after 48 hours for the 1 % wt sample.

### Example 10c: N1-dodecylterephthalamide

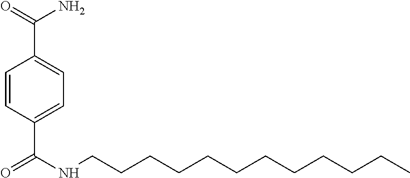 [SMILES: CCCCCCCCCCCCNC(=O)c1ccc(C(N)=O)cc1]

N1-dodecyl-terephthalamide can be prepared by condensation between monoamide of terephthalic acid (15.5 g i.e. 0.1 mol) and laurylamine (18.5 g 0.1 mol) in presence of coupling agents i.e. EDC,HCl (1.2 equ.), HOBt (1.3 equ.) and Diisopropylethylamine (DIPEA—5equ.) in a mixture of THF and DMF (5/1 vol). The mixture was stirred at room temperature overnight (18 h). The mixture was then filtered, washed by THF twice and dried under vacuum to give 26 g of a white solid with a global yield of 80% (purity>95%).

Compositions of 1 wt % and 2 wt % of N1-dodecylterephthalamide in Rapeseed oil were prepared according to the same protocol as described above. Neither samples became transparent and clear after 4 minutes of sonication even extended to 10 minutes. No Gel formation was observed for either after 24 hours and up to a week after sample preparation. Samples were both white liquid slurries.

1. A compound of formula (I): 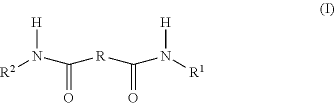
wherein:
R¹ or R² is hydrogen or a linear, branched or cyclic, saturated or unsaturated, hydrocarbon chain having from 1 to 40 carbon atoms, with the proviso that either R¹ or R² is hydrogen,
R is cyclic or branched, saturated or unsaturated, hydrocarbon aliphatic chain having from 2 to 15 carbon atoms.

2. (canceled)

3. The compound according to claim 1, wherein R¹ or R² is a linear or branched, saturated or unsaturated, aliphatic hydrocarbon group having from 2 to 40 carbon atoms.

4. A process for manufacturing the compound of formula (I) according to claim 1, said process comprising the reaction between an alkylamine and a reactant selected from the group consisting of an imide, a diacid, a diester, a primary diamide, an ester amide, and an acid ester.

5. The process according to claim 4, comprising the reaction between at least one alkylamine of formula (II) 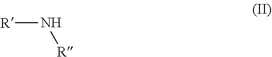
and at least one reactant selected from the group consisting of:
a cyclic imide of formula 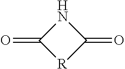
a diacid of formula (IV): 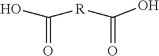
a diester of formula (V): 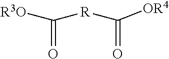
a primary diamide of formula (VI): 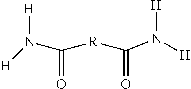
an ester amide of formula (VII): 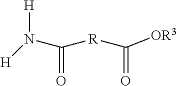
and an acid ester of formula (VIII): 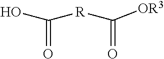
wherein:
R has the same meaning as above in formula (I),
R′ is either R¹ or R², R¹ and R² having the same meaning as above in formula (I),
R″ is either hydrogen or a hydrocarbon chain having from 1 to 40 carbon atoms,
R³ and R⁴ independently to each other, represent a hydrocarbon aliphatic chain, saturated or unsaturated, linear or branched, having from 1 to 40 carbon atoms.

6. A mixture (S) comprising at least one compound of formula (I) according to claim 1 and at least one solvent, said compound of formula (I) being fully solubilized in said solvent.

7. The mixture according to claim 6, wherein the solvent is a polar solvent selected from the group consisting of alcohols having from 1 to 6 carbon atoms, acetal derivatives having from 2 to 12 carbon atoms, alkyl esters having from 2 to 12 carbon atoms, and mixtures thereof.

8. The mixture according to claim 6, wherein the compound(s) of formula (I) represent(s) from 10% to 50% by weight of the total weight of the mixture.

9. A method comprising using the compound of formula (I) according to claim 1 as a gelling agent.

10. A gel composition comprising a carrier and at least one compound of formula (I) according to claim 1, wherein the compound(s) of formula (I) is (are) self-assembled in said carrier in order to provide jellification thereof.

11. The gel composition according to claim 10, wherein the carrier is selected from the group consisting of mineral oils, vegetable oils, animal oils, synthetic oils, linear and iso-alkanes, diesters having from 8 to 40 carbon atoms, glycerol, ethyl carbonate and dimethyl carbonate.

12. The gel composition according to claim 10, wherein the compound(s) of formula (I) represent(s) from 0.01% to 10% by weight of the total weight of the gel composition.

13. The gel composition according to claim 10, wherein the carrier represents from 50% to 99.99% by weight of the total weight of the gel composition.

14. The gel composition according to claim 10, wherein the gel composition is a battery electrolyte.

15. The gel composition according to claim 10 wherein the gel composition is a cosmetic product.

16. The compound according to claim 3, wherein R is not cyclic.

17. The compound according to claim 16, wherein
R is a hydrocarbon aliphatic chain comprising a hydrocarbon main chain having from 1 to 14 carbon atoms and a side chain having from 1 to 6 carbon atoms, and
R¹ or R² is a linear or branched, saturated or unsaturated, aliphatic hydrocarbon chain having from 4 to 32 carbon atoms.

18. The compound according to claim 17, wherein
R is a hydrocarbon aliphatic chains comprising a hydrocarbon main chain having from 2 to 8 carbon atoms and a side chain having from 1 to 4 carbon atoms, and
R¹ or R² is a linear or branched, saturated or unsaturated, aliphatic hydrocarbon chain having from 5 to 24 carbon atoms.

19. The compound according to claim 18, wherein R is selected from —CH(CH₃)—CH₂—; —CH₂—CH(CH₃)—; —CH(CH₃)—CH₂—CH₂—; —CH(CH₂—CH₃)—CH₂— and —CH₂—CH₂—CH(CH₃)—.

20. The compound according to claim 18, wherein R¹ or R² is linear.

21. The compound according to claim 18, wherein
R is selected from the group consisting of —CH(CH₃)—CH₂—; —CH₂—CH(CH₃)—; —CH(CH₃)—CH₂—CH₂—; —CH(CH₂—CH₃)—CH₂— and —CH₂—CH₂—CH(CH₃)—, and
R¹ or R² is selected from the group consisting of pentyl, hexyl, octyl, decyl, dodecyl, tetradecyl, palmityl, stearyl, oleyl, linoleyl, linolenyl, arachidyl and behenyl groups.
